(19) United States
(12) Patent Application Publication
(10) Pub. No.: US 2006/0019251 A1
(43) Pub. Date: Jan. 26, 2006

(54) Method for the qualitative and quantitative determination of genetic material

(75) Inventor: Diana Hormisch, Speyer (DE)

Correspondence Address: SWANSON & BRATSCHUN L.L.C., 1745 SHEA CENTER DRIVE, SUITE 330, HIGHLANDS RANCH, CO, 80129, US

(73) Assignee: Bezirksverband Pfalz, 67653 Germany (DE)

(21) Appl. No.: 10/505,984

(22) PCT Filed: Feb. 25, 2003
(86) PCT No.: PCT/EP03/01921

§ 371 Date: Jul. 12, 2005

(30) Foreign Application Priority Data
Feb. 27, 2002 (DE) 102 08 399.1

(51) Int. Cl. C12Q 1/68

(52) U.S. Cl. 435/6

(57) ABSTRACT

The invention relates to a method for qualitatively and quantitatively determining genetic material which contains a known target sequence which is characteristic for the material and which can be present alongside, or in a mixture with, other genetic material, in which a suspension which contains the genetic material possessing the target sequence and, where appropriate, other genetic material is treated with (1) a molar excess of a first oligonucleotide probe whose nucleotide sequence is complementary to the target sequence and, where appropriate, (2) with a molar excess of a second oligonucleotide probe whose nucleotide sequence is complementary to a nucleotide sequence which is present in all the components of the total genetic material to which the quantification is to relate, the total genetic material, insofar as it is double-stranded, is rendered single-stranded, the first oligonucleotide probe is hybridized with the target sequence and, where appropriate, the second oligonucleotide probe is hybridized with the nucleotide sequence which is present in all the components of the genetic material, the concentration(s) of the unhybridized first and, where appropriate, second oligonucleotide probes is/are determined in the suspension and the proportion of the genetic material possessing the target sequence in the total genetic material to which the quantification is to relate is elucidated from the concentration or concentrations.

The invention relates to a method for qualitatively and quantitatively determining genetic material which can be present alongside, or in a mixture with, other genetic material.

### 1. PRIOR ART

Methods for quantitatively detecting particular organisms play an important role in a variety of fields, for example in the food industry, in agriculture, in quality control, in public health and in research. They are used, for example, in foodstuff and feedstuff diagnostics for determining pathogenic organisms at the same time as apathogenic organisms and for determining the contents of individual animal, plant or microbial components in foodstuffs or feedstuffs. In biotechnology, such methods are required, for example, for determining the expression of genes, while in medicine they are: required, inter alia, for determining the frequency of particular cells in tissue material. In microbiology and in the hygiene sector, methods are likewise employed for determining specific microorganisms (molds, yeasts, bacteria and viruses) at the same time as other microorganisms.

In the food sector and in agriculture, ever greater importance is being attached to genetically modified organisms (GMO). On account of the risks which may be associated with them, they are the subject of legal regulations which include, for example, the obligation to make a declaration when particular highest quantities are exceeded, or even prohibitions. There is therefore a need for a method which can be used to monitor the observance of these regulations, for example which makes it possible to determine the proportion of transgenic corn in corn used in feedstuffs or the content of soybean flour in flour prepared from transgenic soybeans.

Known methods for quantitatively or semiquantitatively determining genetic material are estimation of quantity in an agarose gel following competitive PCR and subsequent staining with ethidium bromide, real-time PCR using fluorescent dyes and commercially available test systems which, by coupling PCR and ELISA detection, enable DNA sequences to be specifically amplified and detected.

With the exception of real-time PCR (Hübner, P. et al, J. AOAC Int. 84 (6) (2001), 1855-1864), the results obtained with these methods are at best semiquantitative (Briggs D., International Biotechnology Laboratory 4/2002, 10). In the case of real-time PCR using fluorescent dyes, problems in the quantification (e.g. quenching) can arise depending on the template and the gene sequence which is to be detected. In addition to this, the analyses are cost-intensive due to the fluorescent dyes which have to be used (Pöpping, B, International Laboratory, 31/4 (2001), 23-29).

In all the methods which have been mentioned, an enzymic reaction is required for quantifying the genetic material. If inhibitory factors, which impair the activity of the enzyme, are present in the genetic material to be investigated, it is not then possible to perform any reliable quantitative determination.

WO 97/38132 describes a method for analyzing nucleic acid. One of the two embodiments disclosed aims at detecting differences from the usual order in the order of nucleotides in known DNA sequences (target sequences). For this, the target sequence is advantageously immobilized on a solid phase, rendered single-stranded and incubated with a mixture of short-chain oligonucleotides which are entirely or partially complementary to the target sequence. The solid phase is separated from the liquid phase and, in the latter, the unhybridized fractions of the nucleotide probes employed are hybridized with immobilised, single-stranded DNA sequences which are complementary to the nucleotide probes and the quantity of nucleotide probes bound to these complementary, immobilized DNA sequences is determined. An increase in the concentration of one or more probes indicates a difference in the order of nucleotides in the target sequence.

In another embodiment, the WO 97/39132 method is suitable for quantifying amplified nucleic acid products. For this, the target sequence is amplified together with a known quantity of a competitive nucleic acid. The mixture of the amplificates is immobilized on a solid phase, rendered single-stranded and incubated with (i) a probe which is complementary to the target sequence, (ii) a probe which is complementary to the competitive nucleic acid and (iii) alternatively, a third probe which is complementary both to the target sequence and to the competitive nucleic acid. After that, the unhybridized probes, which have remained in the solution, are determined as previously described. The quantity of the amplified target sequence, and consequently the quantity of the target sequence which was originally present as well, are determined from the concentration values.

### 2. OBJECTS OF THE INVENTION

One object of the invention is to provide an advantageous method for qualitatively and quantitatigely determining genetic material which contains a characteristic, known nucleotide sequence and can be present alongside, or in a mixture with, other genetic material. Another object of the invention is to provide a method for determining the concentration of a genetic material in a suspension or solution. Another object of the invention is to provide a method which avoids imprecise and elaborate quantification of labeled PCR amplificates and therefore makes it possible to perform a reliable, rapid and economic quantitative determination. Further objects and advantages of the invention are disclosed in the following description.

### 3. BRIEF DESCRIPTION OF THE INVENTION

The invention relates to a method for qualitatively and quantitatively determining genetic material which contains a characteristic, known nucleotide sequence (termed “target sequence” in that which follows) and is present alongside, or in a mixture with, other genetic material. The method is characterized in that a suspension or solution which contains the genetic material possessing the target sequence and also other genetic material (together termed “total genetic material” in that which follows) is treated with (1) a molar excess of a first oligonucleotide probe (termed “first probe” in that which follows) whose nucleotide sequence is complementary to the target sequence and (2) with a molar excess of a second oligonucleotide probe (termed “second probe” in that which follows) whose nucleotide sequence is complementary to a nucleotide sequence (termed “control sequence” in that which follows) which is present in all the components of the total genetic material to which the quantification is to relate, the total genetic material, insofar as it is double-stranded, is rendered single-stranded, the first probe is hybridized with the target sequence and the second probe is hybridized with the control sequence, the concentrations of the unhybridized first and second probes are determined in the suspension or solution and the proportion of the genetic material possessing the target sequence in the total genetic material to which the quantification is to relate is elucidated from the concentrations.

In a first embodiment, the method according to the invention operates with two probes and gives the quantitative proportions of the genetic material to be determined and of a second genetic material which is present alongside it or in a mixture with it, for example as percentages by weight based on the sum of the two genetic materials.

A second embodiment of the method operates with only one probe and serves to determine the concentration of genetic material in a suspension or solution, for example as an organism count per unit volume. Accordingly, this embodiment is a method for quantitatively determining the concentration of genetic material which contains a characteristic known nucleotide sequence (termed “target sequence” in that which follows) and can be present alongside, or in a mixture with, other genetic material. The method is characterized in that a suspension which contains the genetic material possessing the target sequence and also, where appropriate, other genetic material is treated with a molar excess of only one oligonucleotide probe (the “first oligonucleotide probe” of the previously described first embodiment) whose nucleotide sequence is complementary to the target sequence, the genetic material, insofar as it is double-stranded, is rendered single-stranded and the probe is hybridized with the target sequence, the concentration of the unhybridized probe is determined in the suspension or solution and the concentration of the genetic material in suspension is elucidated from this concentration.

### 4. FEATURES, ADVANTAGES AND POSSIBLE APPLICATIONS OF THE METHOD

Both the described embodiments of the method according to the invention determine the quantity(ies) or concentration(s) of the unhybridized probe(s). The difference between the quantity of probe employed and the quantity of unhybridized probe correspond to the genetic material which is in each case to be determined.

The method according to the invention makes it possible to both qualitatively detect and quantitatively determine genetic material. In its first embodiment, it is directed toward quantifying genetic material, just like the second embodiment of the method described in WO 97/38132. In contrast to the known method, the genetic material does not have to be prepared by amplification. Another difference is that, while a competitive nucleic acid has to be used as an auxiliary in the known method, the method according to the invention makes do without any such auxiliary. Furthermore, according to the invention, two probes, one of which is hybridized with the target sequence while the other is hybridized with a control sequence which is present in both genetic materials, are used to determine the quantitative proportions of two genetic materials. By contrast, the known method operates with (i) a probe which is complementary to the target sequence, (ii) a probe which is complementary to the competitive nucleic acid, and (iii) only alternatively, a third probe which is complementary to both the target sequence and the competitive nucleic acid. This method consequently either uses no probe which is complementary to both the target sequence and the competitive sequence or else requires, in addition to such a probe, two further probes.

The second embodiment of the method according to the invention is aimed at determining the concentration of a genetic material in a suspension or solution and simply because of this differs from the first embodiment of the method described in WO 97/38132, the purpose of which first embodiment is to elucidate differences in the order of the nucleotides in genetic material. Consequently, the document does not contain the teaching to deduce the concentration of the genetic material in the initial suspension or solution from the concentrations of the unhybridized probes.

The method according to the invention also offers a number of advantages as compared with the other previously described methods in accordance with the prior art. Using biosensors to achieve a label-free detection, for the purpose of determining the probe concentrations after hybridization, avoids the elaborate and imprecise quantification achieved when using labeled PCR amplificates. Miniaturization and automation makes it possible to perform simultaneous measurements. Other advantages are the ready regenerability of the biosensors, smaller sample and reagent volumes and s marked facilitation of the work involved. The high-affinity interactions between the biomolecules make it possible to perform quantitative measurements using genetic materials which are prepurified to a lesser degree. There is no need for expensive reagents, as are required for the previously mentioned fluoresence detection. Furthermore, the time required for the methods according to the invention is less than in the case of known methods.

According to the invention, it is possible for example, to draw conclusions with regard to gene, transcript, expression, cell or organism frequencies and organism distributions or product compositions. In this connection, it is possible, for example, to determine DNA alongside DNA, RNA alongside RNA and DNA alongside RNA. In particular, the method according to the invention is, for example, outstandingly suitable, in its two embodiments, for achieving the analytical objects described under the above item 1. In particular, it solves the following problems, for example:
Identifying and quantifying DNA fragments after a DNA mixture has been fractionated by gel electrophoresis. In this case, no other genetic material apart from the genetic material having the target sequence is present.
Monitoring the concentration of cells in biotechnological methods. In this case, other genetic material in addition to the material having the target sequence may be present.
Determining the concentration of DNA-containing or RNA-containing biological material in blood or other liquids.
Determining the proportion of a particular bacterial species in a bacterial population, for example of a particular lactic acid bacterial species in the total population of lactic acid bacteria. The target sequence is DNA. If it is only the number of the particular lactic acid bacteria per unit volume which is to be determined, only the first probe is required. If the proportion of the particular lactic acid bacteria in the total lactic acid bacterial population is to be determined, the second probe is also required. The control sequence is then likewise DNA. The total genetic material is derived from genetically different, even if to a large extent identical, organisms which possess a control sequence in common.
Determining the content of soybean flour in GMO soybean or of corn flour in GMO corn. The target sequence and the control sequence are DNA and the total genetic material is once again derived from organisms which are genetically different even if to a large extent identical.
When investigating plasmid frequency in a bacterial cell, the target sequence is plasmid-encoded and the control sequence is chromosomally encoded and two DNA sequences which are derived from the same organism are determined.
If the number of RNA copies of a gene is determined in the cell plasm, in order to obtain information as to how active the gene is at the time in question or under the prevailing conditions, RNA and DNA are compared and the genetic material is likewise derived from only one organism.
When determining the quantitative proportion of RNA copies of genes, RNA is compared with RNA and the genetic material is once again derived from only one organism.

### 5. DETAILED DESCRIPTION OF THE INVENTION

The method according to the invention is initially described with reference to the first of the two embodiments.

5.1 The Genetic Material

In this present document, the term genetic material is used synonymously with nucleic acid. The genetic material can be single-stranded or double-stranded. It can be DNA, RNA or PNA or contain other modified base sequences and is as a rule derived from biological material which can consist of only one organism or can contain several, different organisms. The biological material includes, for example, viruses, bacteria, fungi, yeasts, plants and animals, or parts thereof or their products or processing products. The total genetic material on which the method according to the invention is based can be obtained from the corresponding biological material in a customary manner. Suitable known methods are used for isolating or enriching the target sequence and/or the control sequence, depending on the objective.

The target sequence and the control sequence are nucleotide sequences which have a known order of bases. They can be a gene, plasmid, RNA molecule or PNA molecule or another modified base sequence. Fragments of these constructs can also be the target sequence or control sequence.

It is possible for the quantification to only relate to a part of the total genetic material. If, for example, the proportion of GMO soybean is determined in a feed meal, this proportion can be related to the total feed meal or, as is frequently desired, to the proportion of soybean meal in the feed meal. In this case, a control sequence which is only present in GMO corn and natural corn, and a corresponding second probe, are chosen.

5.2 Hybridizing the Probes to the Target Sequence and the Control Sequence

The method according to the invention is based on a buffer solution which contains the total genetic material in suspension or in solution. The suspension or solution is treated with molar excesses of the first and second probes. “Molar excess” means that there is at least one molecule of probe for each of the target sequences or control sequences in the total genetic material. The probe quantities which are required for an excess can be determined by means of preliminary experiments beginning with small quantities of the probes. A deficiency is indicated by the fact that the probe can no longer be detected after the hybridization. The concentrations are then increased until a clearly measurable signal is obtained. The optimal magnitude of the excess of the probes depends crucially on the number of target sequences or control sequences which are present in the suspension or solution and has to be determined from case to case.

The two probes each contain a single-stranded oligonucleotide sequence (e.g. composed of DNA, RNA, PNA or other modified base sequences). They expediently comprise from 6 to 70 nucleotides, in particular from 13 to 25 nucleotides, and are complementary to the whole, or to a part, of the target sequence or the control sequence, respectively. The order in which the nucleotides are arranged in the target sequence and the control sequence has to be known so that the probes can be prepared in the complementary base order using customary methods. Probes containing the desired order of magnitude can be obtained commercially. Like the genetic material, they are advantageously used suspended or dissolved in buffer solution.

Insofar as the total genetic material is double-stranded, it has to be rendered single-stranded prior to the hybridization. For this, the solution is expediently heated at a temperature of from 95 to 100° C. for from 5 to 20 minutes and the DNA is then allowed to cool slowly down to room temperature with the probes hybridizing to the target sequence and control sequence, respectively. Depending on the hybridization conditions and the probe constitution (order of nucleotides and probe length), the hybridization is generally complete after from 1 to 120 min. After the hybridization, the concentration of the unhybridized probes are determined.

5.3 Determining the Concentrations of the Two Unhybridized Probes

One of the various label-free detection methods which have been specially developed for analyzing biomolecular interactions is advantageously used for determining the concentration of the unhybridized probe molecule. In Biosensoric 1, Fundamental Aspects (1997), edited by Scheller, F. W., Schubert, F. and Fedrowitz, J., Birkhäuser Verlag, Basle, Switzerland, Bier, F. F. et al. provide a review of the various methods of bioaffinity analysis. A biosensor is an arrangement in which a biologically sensitive element is coupled to a physical transducer. Using this arrangement, the highly specific reaction of the probe molecules with specific biomolecules is translated into a measurable signal by means of the transducer. Electrochemical methods and transducers are described by Ullman, M., Synthese von Peptiden zur Verwendung in einem Biosensor [Synthesis of peptides for use in a biosensor] (1998), Reihe Chemie [Chemistry series], ad fontes-Verlag, ISBN 3-932681-08-8, while piezoelectric and/or mass-sensitive methods are described by Liebau, M., Resonante Quarzsensoren [Resonant quartz sensors], dissertation, Halle-Wittenberg University (1998) and optical methods are described by Brecht, A. and Gauglitz, G., Optical probes and transducers, Biosens & Bioelectron, 10 (1995) 923-936. In all these methods, the analyte, that is the probe molecules, are present in solution while the other reaction reaction partner is immobilized, as ligand, on a surface.

Optical methods are very sensitive and are therefore preferred. One of these methods uses the phenomenon of surface plasmon resonance (SPR). The method is described in detail in the publication by K. Nagata and H. Handa, Real-time Analysis of Biomolecular Interactions: Applications of BIACORE, Springer-Verlag Tokyo Berlin Heidelberg New York, ISBN 4-431-70289-X, in particular on pages 1 to 83. The basic principle of all SPR sensors is the phenomenon of the optical excitation of the oscillation of electrons in a thin metal film (Kretzmann, E., and Raether, H., Radiative decay of non-radiative surface plasmons by light, Z. Naturforsch., Vol 23a (1968), 2135). The oscillations, what are termed polaritons or plasmons, which are formed by interaction with the photons diffuse out in a wave-like manner over a few micrometers in the metal layer. The oscillations are associated with an evanescent field which extends through the phase boundary into the environment. Monochromatic light is beamed in through a prism, which, from the rear side of the sensor chip, presses against a glass substrate, and the angle spectrum of the reflected light is subsequently read off. Since light of a particular wavelength and of a particular incident beam angle is absorbed for activating the SPR effect, an intensity minimum appears in the reflection. If small changes in the mass loading now occur on the metal surface of the sensor chip, for example as a result of the accumulation of proteins or nucleic acids, the molecules then interact with the electromagnetic evanescent field. The result of this interaction is a detectable change of the intensity minimum in the reflection angle spectrum, with this change correlating with the increase in mass (Kuhlmeier, D., Nachweis von Nukleinsäure-Wechselwirkungen mit optischen Biosensoren [Detection of nucleic acid interactions with optical biosensors], dissertation, Technical University, Brunswick (2000)). In this way, interactions of molecules with the evanescent field can be detected in an extremely sensitive manner.

The company Biacore AB, Rapsgatan 7, S-754 50 Uppsala, Sweden, sells a variety of models of instruments for measuring by the SPR method, including the analytical software. The Biacore measuring system records the sensor signal in what are termed resonance units (RU), with it being arbitrarily stipulated that 1000 RUs correspond to an accumulation of 1 ng/mm² (Stenberg, E. et al, Quantitative determination of surface concentration, J. Coll. Interf. Sci., 143, 513-526 (1991)). On the other hand, 1000 RUs correspond to a deflection of the reflection angle by 0.1°. The data are presented in a graph having an ordinate (RUs) and an abscissa (time).

In order to detect the given unhybridized probe, an appropriate ligand is immobilized on the sensor chip. Depending on the constitution of the target sequence and/or control sequence, this ligand comprises DNA, RNA, PNA or similar base sequences which are complementary to the base sequences of the probes (Kilsa Jensen et al, Kinetics for Hybridization of Peptide Nucleic Acids, Biochemistry, 36, 5072-5077 (1997)) and, in addition, also carry functional groups for an immobilization, on the sensor chip, which is based on covalent bonding or on physical interactions. They hybridize with the probe molecules as a result of specific base pairing by way of hydrogen bonds. An example of a functional group which is suitable for immobilizing the ligand molecules is biotin, provided streptavidin is doped on the sensor chip. The abovementioned company Biacore AB, for example, sells such sensor chips. Biotin group-containing oligonucleotides possessing the desired nucleotide sequences can be obtained commercially.

For the purpose of measuring the concentration of the unhybridized probe molecules, the suspension flows, at a defined temperature of, for example, 25.0° C., and at a defined speed, for example of 25 μl/min, past the sensor chip, or consecutively or in parallel streams past the sensor chips, with each sensor chip, or each measurement site on a sensor chip, being loaded with an oligonucleotide which is complementary to the probe to be measured.

After the measurement, the sensor chips are regenerated, and prepared for a new measurement, by using a suitable solution (expediently a 50 mM NaOH solution; 10 sec) to release the bound probe molecules from the immobilized oligonucleotides under conditions which leave the latter on the sensor chip.

5.4 Determining the Proportion of the Genetic Material Having the Target Sequence in the Total Genetic Material

In order to determine the proportion of the target sequence-containing genetic material in the total genetic material or in that part of the genetic material to which the quantification is to relate, the measuring arrangement has to be calibrated, with this expediently being done prior to the measurement. For the calibration, solutions which contain known, varying concentrations of first or second probe, but which do not contain any genetic material or contain the total genetic material without a hybridization step, are caused to flow, under measurement conditions (temperature, flow rate) past the sensor chips and the SPR signal is in each case measured. The connection between the concentrations of the probe molecules and the strength of the SPR signal can be depicted graphically in a calibration curve.

The measurements of the concentration of the first probe after the hybridization of the target sequence and of the concentration of the second probe after the hybridization with the control sequence result in two values. If, after the hybridization, the concentration of the first probe has remained unchanged as compared with the concentration without hybridization, no genetic material possessing the target sequence was present in the total genetic material. If, after hybridization, the concentration of first probe has decreased as compared with the concentration without hybridization, genetic material possessing the target sequence has thereby been qualitatively detected. The quantitative determination, that is the proportion of the target sequence-containing genetic material in the total genetic material containing the control sequence ensues from comparing the measured concentrations of the first and second probes, in each case with and without hybridization.

5.5 The Method in its Second Embodiment

The description of the method in accordance with the first embodiment also applies, mutatis mutandis, to the second embodiment. However, there is no control sequence, no second oligonucleotide probe and no sensor chip on which a ligand which is complementary to the second oligonucleotide probe is immobilized. For example, the concentration of viruses in blood can be determined by selecting a viral target sequence, bringing a defined volume of virus-containing blood into contact, under hybridization conditions, with a defined molar excess of an oligonucleotide probe which is complementary to the target sequence (“probe hybridization”), immobilizing, as ligand, an oligonucleotide sequence which is complementary to this oligonucleotide probe, and causing the suspension or solution which contains the hybridized target sequence and the unhybridized nucleotide probes to flow past the immobilized oligonucleotide sequences (“ligand hybridization”). A previously determined calibration curve is used to assign the detector signal to a particular molar quantity of oligonucleotide probe, with this signal giving, by subtraction, the molar quantity of oligonucleotide probe which has been consumed by the probe hybridization. This molar quantity is equivalent to the molar quantity of viral target sequence and consequently also of virus. Dividing by the defined volume gives the concentration of virus.

### 6. SPECIAL DETECTION METHODS

6.1 Hybridizing the Probes with the Genetic Material

In a variant of the first embodiment of the method according to the invention, the hybridization of the first and second probes is carried out in individual steps using constituent quantities of the same genetic material. Alternatively, the first and second probes can be hybridized with the genetic material in one and the same reaction mixture and the excess, unbound probe molecules can then be determined.

6.2 Receptor/Target Molecule Reactions as Signal-Emitting Bioreactions

It was previously stated that the unhybridized oligonucleotide probes (as target molecules) are bound by immobilized oligonucleotides (as receptors) by reason of specific base pairing. This binding principle can be replaced in a variety of ways:

6.2.1 Thus, instead of using an oligonucleotide, it is possible to use an immobilized antibody (as receptor) which is directed against an antigen (as target molecule) which, for its part, is bound to the probe molecule. Thus, anti-Dig (Dig=digoxigenin) can be immobilized on the sensor chip and the probe can be linked to the Dig antigen. The probe molecules are then removed from the solution by means of an antigen/antibody reaction using the immobilized antibody and concentrated on the boundary surface in a signal-emitting manner.

6.2.2 It is furthermore possible, instead of immobilizing an oligonucleotide, as receptor, to immobilize an antigen (e.g. Dig) on the sensor chip and to link the probe to the same antigen. After the hybridization, the unhybridized Dig-modified probe molecules are treated with a defined excess of antibody (anti-Dig). The excess antibody, which is not bound in this connection, enters, as target molecule, into an antigen/antibody reaction with the immobilized antigen receptor, thereby in turn generating a biosignal.

6.3 Method with Preceding PCR

If, in an individual case, the quantity of total genetic material available is too small for a sufficiently accurate determination, or the total genetic material only contains very small quantities of genetic material possessing the target sequence, the quantity of the material can be increased, for example, by means of PCR. The determination, by means of SPR, of the probes remaining after the hybridization (and consequently the determination of the probes consumed by hybridization) is considerably more accurate than the determination of amplificate on a gel using the fluorescence method.

6.4 Increasing the Sensitivity or Lowering the Detection Limit

As previously explained, the strength of the biosignal depends on the concentration of probe molecules at the boundary surface. However, it also depends, for the same concentration, on their molar masses. The biosignal, and thus the sensitivity of the method, can be increased substantially by increasing the molar masses of the probe molecules:

6.4.1 For example, the molar mass of antigen-modified probes (see 6.2.2) can be increased if an antibody which is directed against the antigen is allowed to act on them and, where appropriate, the molecule, which has been enlarged in this way, is further enlarged using another, second antibody, which is directed against the first antibody. For example, the molar mass of a Dig-modified probe can be increased with anti-Dig and further increased with anti-anti-Dig.

6.4.2 The molar masses of probes, which may, where appropriate, be antigen-modified, can be increased if the number of nucleotides is increased from about 20, which is the quantity which is desirable for reliable recognition of the target sequence, up to about 70.

6.5 Universal Chip Strategy for Different Probes

6.5.1 If the antigen/antibody-reaction detection system as described in 6.2.1, 6.2.2, 6.4.1 or 6.4.2 is being used and both the first and the second probe are linked to the same antigen, it is possible to use the same sensor chip for both measurements.

6.5.2 The detection system which is based on specific base pairing can be generalized by initially carrying an (immobilizing) prehybridization step using a binding mediator which possesses a first nucleotide sequence, which is complementary to the nucleotide sequence which was immobilized on the sensor chip, and a second nucleotide sequence, which is complementary to the target sequence or to the control sequence. Use is made of first and second probes which contain a nucleotide sequence which is complementary to the second nucleotide sequence of the binding mediator which is still free after the prehybridization and the excess first and second probes are hybridized with the immobilized binding mediator. The first hybridization step provides information about the quality of the sensor chip in regard to the number of sites to be occupied. Stringent washing steps can be used to release the probe molecules and the binding mediator from the immobilized receptor such that the sensor chip can bind other probe molecules using a different binding mediator.

6.6 Controlled Determinations

In order to check the results obtained with a particular first probe and a particular second probe, it is additionally possible to use alternative first and/or second probes which hybridize with other base sequences in the target sequence or the control sequence. The alternative probes can more or less overlap with the probes to be checked or else have a completely different base sequence.

Suitable antigens and antibodies, as well as probes which possess the desired nucleotide sequence and which are linked to antigens or antibodies, as mentioned in the above sections of this item 6, can be prepared using customary methods or can be obtained commercially.

The steps listed under item 6 can be taken alternatively and, where appropriately, cumulatively.

### EXAMPLE

Determining GMO Corn in Corn Flour

Preparing the Genetic Material

2 g of the corn flour in which the proportion of GMO corn was to be quantified were used. The DNA was extracted by standard methods (Mühlhardt, C., Der Experimentator: Molekularbiologie [The Experimentor: Molecular biology], 2nd edition (2000), pp. 11-37, Spektrum Akademischer Verlag). Since the target sequence only constituted a small proportion of the total genetic material, it was amplified by PCR in order to enrich it. The essential data for the primers used for the amplification are summarized in Table 1 below.

TABLE 1
PCR primers employed for amplifying the target sequences

| | | | |
|---|---|---|---|
| Designation | Detection target | Base sequences of the PCR primers | Length |

| | | | | |
|---|---|---|---|---|
| 1 | 35S-F | 35 S GMO-CaMV promoter | 5′-CCTACAAATGCCATCATTGCG-3′ | 21-mer |
| 2 | 35S-R | control element | 5′-GGGTCTTGCGAAGGATAGTG-3′ | 20-mer |
| 3 | lvr-1 | Zea mays invertase gene | 5′-CCGCTGTATCACAAGGGCTGGTACC-3′ | 25-mer |
| 4 | lvr-2 | | 5′-GGAGCCCGTGTAGAGCATGACGATC-3′ | 25-mer |

35 cycles of a standard PCR program were carried out using Taq polymerase obtained from Quiagen, D-40721 Hilden, Federal Republic of Germany (Mühlhardt, loc. cit., pages 67-81). The presence of the amplificate was checked by means of agarose gel electrophoresis (Mühlhardt, loc. cit., pages 46-51). It was not possible to detect any nonspecific PCR products.

Hybridization Reaction of the Probes with Target and Control Sequences

One oligonucleotide sequence (=first probe) was derived from the nucleotide sequence of the CaMV promoter (GenBank accession number V00141) (Franck, A. et al, Nucleotide sequence of cauliflower mosaic virus DNA, Cell 21 (1980), 285-294). It possesses a base sequence which is complementary to a target region within the CaMV35S target sequence (see Tab. 2) and enables the target region to be detected in a highly specific manner.

The first probe was added in excess with respect to the expected concentration of the target sequence:

Mixture: 15 μl of a solution of the PCR mixture diluted with 0.5 M NaCl HBS-EP buffer in the ratio 1:10,
20 μl of first probe (2 mM in the total mixture)
35 μl of 0.5 M NaCl HBS-EP buffer

The mixture was incubated at 100° C. for 10 min and then cooled down to room temperature.

Another oligonucleotide sequence (=second probe) was derived from the sequence of the Zea mays invertase gene (GenBank accession number U16123) (Xu, J. et al, Plant Physiol. 108(3), 1293-1294). It possesses a base sequence which is complementary to a target region within the corn-typical invertase control sequence (see Table 2) and enables the control sequence to be detected in a highly specific manner. This second probe was hybridized with the following experimental mixture, as Is described for the first probe:

Mixture: 15 μl of solution of the PCR mixture diluted with 0.5 M NaCl HBS-EP buffer in a ratio of 1:10,
20 μl of second probe (4 mM in the total mixture)
35 μl of 0.5 M NaCl HBS-EP buffer
   Preparing the Specific Sensor Surfaces

The measurements were carried out using a Biacore Q from Biacore AB, see above. The sensor chip which was used was precoated with streptavidin (Biochip SA; Biacore AB, see above). A biotin-labeled oligonucleotide which was complementary to the two probes (MWG-Biotech AG, D-85560 Ebersberg) was in each case immobilized, as ligand, on the streptavidin surface (Wood, S. J., Microchemical Journal 47 (1993), 330-337). The essential data for the ligands employed are listed in Table 2 below.

TABLE 2

Oligonucleotide sequences employed for detecting the
reduction in the first and second probes

| | | Detection | | |
|---|---|---|---|---|
| Designation | Function | target | Length | Base sequence |
| 35S probe | Ligand immobilized on the biochip | 35S GMO | 21-mer | 5′-biotin-AAG AAA AAG |
| compl. | by means of 5′-biotinylation | element | | GTG CTA CGA GGA-3′ |
| | (in its base sequence, complemen- | | | |
| | tary to the 1^{st} probe) | | | |
| 35S probe | Analyte (= 1^{st} probe) | | 21-mer | 5′-TCC TCG TAG CAC |
| | | | | CTT TTT CTT-3′ |
| Ivr probe | Ligand immobilized on the biochip | Reference | 20mer | 5′-biotin-CAA CCG GAG |
| compl. | means of 5′-biotinylation | gene, inver | | CAT GCC CAC TA-3′ |
| | (in its base sequence, complemen- | tase gene | | |
| | tary to the 2^{nd} probe) | | | |
| Ivr probe | Analyte (= 2^{nd} probe) | | 20-mer | 5′-TAG TGG GCA TGC |
| | | | | TGC GGT TG-3′ |

A 0.5 M NaCl HBS-EP buffer (Biacore AB, see above) was used as running buffer for immobilizing the given ligand. The flow rate was 30 μl/min. After the sensor chip had been conditioned with three one-minute injections of 1 M NaCl in 50 mM NaOH solution, in each case 60 μg/ml of biotinylated oligonucleotides were injected and immobilized on the surface of the sensor chip.

Determining the Unhybridized First Probe

The reaction of the first probe with the immobilized ligand 35S probe compl. was calibrated. The flow rate of the running buffer (see above) was 25 μl/min. The sensor chip signal difference, as measured in resonance units (RU), between the detected base line (only buffer) and the plateau reached after injecting the sample was utilized for determining the quantity of unbound first probe (Wood, loc. cit.). When 30 μl of the sample mixture were injected without any hybridization reaction, the signal difference was 685.2 RUs, corresponding to a first probe concentration of 1.97 mM. When 30 μl of the sample mixture were injected after the hybridization reaction, the signal difference was 442 RUs, corresponding to a concentration of 1.16 mM. Consequently, 1.97 mM minus 1.16 mM=0.81 mM of the first probe had hybridized with the target sequence; this corresponds to 0.81 mM of target sequence.

Following on from the hybridization reaction on the sensor chip, the chip surface was freed from hybridized probe molecules by injecting 15 μl of 1 M NaCl in 50 mM NaOH solution and then neutralized with running buffer.

Determining the Unhybridized Second Probe

The determination was carried out in analogy with determining the unhybridized first probe. The total genetic material was obtained by amplifying genetic material from 2 g of corn flour by PCR in 35 cycles using the primers Ivr-1 and Ivr-2 as shown in Table 1. The base sequences of the second probe and of the complementary ligand are shown in Table 2. Measurement of the sample mixture without any hybridization reaction gave a signal difference of 601 RUs, corresponding to a concentration of second probe of 4.30 mM. Measuring the sample mixture with hybridization gave a signal difference of 366 RUs, corresponding to a concentration of second probe of 2.49 mM. Consequently, 4.30 mM minus 2.49 mM=1.81 mM of the second probe were consumed by hybridization, corresponding to 1.81 mM of control sequence.

Determining the Proportion of GMO

The 1.81 mM of control sequence are set equal to 100%. The 0.81 mM of target sequence then correspond to a proportion of GMO on the corn flour of 44.75%.

1. A method for qualitatively and quantitatively determining genetic material which contains a known target sequence which is characteristic for the material and which is present alongside, or in a mixture with, other genetic material, characterized in that a suspension which contains the genetic material possessing the target sequence and also other genetic material is treated with (1) a molar excess of a first oligonucleotide probe whose nucleotide sequence is complementary to the target sequence and (2) with a molar excess of a second oligonucleotide probe whose nucleotide sequence is complementary to a nucleotide sequence which is present in all the components of the total genetic material to which the quantification is to relate,
the total genetic material, insofar as it is double-stranded, is rendered single-stranded,
the first oligonucleotide probe is hybridized with the target sequence and the second oligonucleotide probe is hybridized with the nucleotide sequence which is present in all the components of the genetic material,
the concentrations of the unhybridized first and second oligonucleotide probes are determined in the suspension and the proportion of the genetic material possessing the target sequence in the total genetic material to which the quantification is to relate is elucidated from the concentrations.

2. The method as claimed in claim 1 for quantitatively determining the concentration of genetic material which contains, as target sequence, the characeristic, known nucleotide sequence and can be present alongside, or in a mixture with, other genetic material, characterized in that
a suspension which contains the genetic material possessing the target sequence and also, where appropriate, other genetic material is treated with a molar excess of only one oligonucleotide probe whose nucleotide sequence is complementary to the target sequence,
the genetic material, insofar as it is double-stranded, is rendered single-stranded and the probe hybridizes with the target sequence,
the concentration of the unhybridized probe is determined in the suspension and the concentration of the genetic material in the suspension is elucidated from this concentration.

3. The method as claimed in claim 1 or 2, characterized in that the genetic material is DNA, RNA, partially DNA and partially RNA, PNA or another modified base sequence.

4. The method as claimed in one of claims 1 to 3, characterized in that the oligonucleotide probes consist of DNA, RNA, PNA or other modified base sequences.

5. The method as claimed in one of claims 1 to 4, characterized in that the concentrations of the two probes are determined using a label-free detection method.

6. The method as claimed in claim 5, characterized in that the detection method is based on surface plasmon resonance.

7. The method as claimed in claims 1 or 3 to 6, characterized in that the hybridization of the first probe and the second probe is carried out in individual steps using constituent quantities of the same genetic material.

8. The method as claimed in claims 1 or 3 to 6, characterized in that the hybridization of the first probe and the second probe with the genetic material is carried out in one and the same reaction mixture and the excess, unbound probe molecules are then determined.

9. The method as claimed in one of claims 1 to 8, characterized in that the first probe and, where appropriate, the second probe are bound, after the hybridization, by immobilized oligonucleotides as the result of specific base pairing.

10. The method as claimed in claim 9, characterized in that the immobilized ligand is an antibody which is directed against an antigen which is also bound to the probe molecules.

11. The method as claimed in claim 9, characterized in that the immobilized ligand is an antigen, the probe is linked to the same antigen, the unhybridized antigen-modified probe molecules are treated with an excess of antibody and the excess antibody is bound by means of an antigen/antibody reaction to the immobilized antigen on the sensor chip.

12. The method as claimed in one of claims 1 to 11, characterized in that it is preceded by a PCR.

13. The method as claimed in one of claims 1 to 11, characterized in that the molar mass of the first and/or second probe is increased.

14. The method as claimed in claim 13, characterized in that the molar mass of antigen-modified probe is increased by allowing an antibody which is directed against the antigen to act on them and, where appropriate, the molar mass of the molecule, which has been increased in this way, is further increased using another, second antibody which is directed against the first antibody.

15. The method as claimed in claim 13, characterized in that the molar mass of probes, which may, where appropriate, be antigen-modified, is increased by increasing the number of nucleotides beyond about 20 to up to about 70.

16. The method as claimed in one of claims 13 to 15, characterized in that both the first probe and the second probe are linked to the same antigen.

17. The method as claimed in one of claims 13 to 16, characterized in that a prehybridization step is carried out using a binding mediator which possesses a nucleotide sequence which is complementary to the nucleotide sequence which is immobilized on the sensor chip and a nucleotide sequence which is complementary to the target sequence and/or to the control sequence, use is made of first and second probes which have a nucleotide sequence which is complementary to the nucleotide sequence of the binding mediator which is still free and the excess first and second probes are hybridized with the binding mediator.

18. The method as claimed in one of claims 5 to 17, characterized in that use is additionally made of alternative first and/or second probes which hybridize with other base sequences of the target sequence or of the control sequence.
